(19) United States
(12) Patent Application Publication
(10) Pub. No.: US 2010/0234438 A1
(43) Pub. Date: Sep. 16, 2010

(54) THERAPEUTIC AGENTS

(76) Inventor: Leifeng Cheng, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 12/304,050

(22) PCT Filed: Jun. 18, 2007
(86) PCT No.: PCT/GB2007/002266

§ 371 Date: Dec. 9, 2008

Related U.S. Application Data
(60) Provisional application No. 60/815,069, filed on Jun. 20, 2006.

(51) Int. Cl. A61K 31/415; C07D 231/12; A61P 3/04; A61P 1/00; A61P 25/28; A61P 25/30; A61P 25/16; A61P 25/08; A61P 25/18; A61P 25/22

(52) U.S. Cl. 514/406; 548/375.1

(57) ABSTRACT

The present invention relates to a 1,5-diphenylpyrazole compound and its use in the treatment of obesity, psychiatric and neurological disorders, to methods for the its therapeutic use and to pharmaceutical compositions containing them.

### FIELD OF INVENTION

The present invention relates to a certain 1,5-diphenylpyrazole compound, to processes for preparing it, to its use in the treatment of obesity, psychiatric and neurological disorders, to methods for its therapeutic use and to pharmaceutical compositions containing it.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties. Pyrazoles having anti-inflammatory activity are disclosed in WO99/64415 and EP 418 845. 1,5-Diarylpyrazole-3-carboxamide derivatives are disclosed as having CB₁ modulatory activity in U.S. Pat. No. 5,624,941, WO03/020217 and EP 656354. 1,5-Diarylpyrazole-3-carboxamide derivatives having a 4-hydroxymethyl substituent are disclosed in US 2004/0192667, EP 0876350 and EP 1,571,147 as having CB₁ modulatory activity. Co-pending application WO2005/080343 discloses 4-[1-(substituted phenyl)-3-[(carboxamido]-1H-pyrazol-5-yl]phenyl 1-alkanesulfonic acid ester derivatives as having CB₁ modulatory activity.

PCT/GB2005/004977 discloses a compound of formula (I)

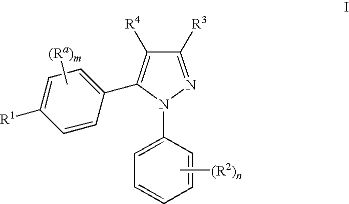

and pharmaceutically acceptable salts thereof, in which

R¹ represents a) a C₁₋₃alkoxy group optionally substituted by one or more of the following i) fluoro ii) a group NR^{e}R^{d} in which R^{e} and R^{d} independently represent H, a C₁₋₆alkyl group or C₁₋₆alkoxycarbonyl group or iii) a 1,3-dioxolan-2-yl group b) R¹ represents a C₄₋₆alkoxy group optionally substituted by one or more of the following i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group c) a group of formula phenyl(CH₂)ₚO— in which p is 1, 2 or 3 and the phenyl ring is optionally substituted by 1, 2 or 3 groups represented by Z, d) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z e) a group of formula (R⁶)₃ Si in which R⁶ represents a C₁₋₆alkyl group which may be the same or different or f) a group of formula R^{b}O(CO)O in which R^{b} represents a C₁₋₆alkyl group optionally substituted by one or more fluoro;

Ra represents halo, a C₁₋₃alkyl group or a C₁₋₃alkoxy group

m is 0, 1, 2 or 3;

R² represents a C₁₋₃alkyl group, a C₁₋₃alkoxy group, hydroxy, nitro, cyano or halo

n is 0, 1, 2 or 3;

R³ represents

a) a group X—Y—NR⁷R⁸

in which X is CO or SO₂,

Y is absent or represents NH optionally substituted by a C₁₋₃alkyl group;

and R⁷ and R⁸ independently represent:

a C₁₋₆alkyl group optionally substituted by 1, 2, or 3 groups represented by W;

a C₃₋₁₅cycloalkyl group optionally substituted by 1, 2, or 3 groups represented by W;

an optionally substituted (C₃₋₁₅cycloalkyl)C₁₋₃alkylene group optionally substituted by 1, 2, or 3 groups represented by W;

a group —(CH₂)ᵣ(phenyl)ₛ in which r is 0,1, 2, 3 or 4, s is 1 when r is 0 otherwise s is 1 or 2 and the phenyl groups are optionally independently substituted by one, two or three groups represented by Z;

a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;

a group —(CH₂)ₜ Het in which t is 0,1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo wherein the alkyl and alkoxy group are optionally independently substituted by one of more fluoro;

or R⁷ represents H and R⁸ is as defined above;

or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl;

or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;

R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;

Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and

W represents hydroxy, fluoro, a C₁₋₃alkyl group, a C₁₋₃alkoxy group, amino, mono or di C₁₋₃alkylamino, a C₁₋₆alkoxycarbonyl group or a heterocyclic amine selected from morpholinyl, pyrrolidinyl, piperidinyl or piperazinyl in which the heterocyclic amine is optionally substituted by a C₁₋₃alkyl group or hydroxyl; and its use in the treatment of obesity, psychiatric and neurological disorders.

The compound 3,3,3-trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester is specifically disclosed in this application. It has now been found that the minus enantiomer of this compound has beneficial properties.

### DESCRIPTION OF THE INVENTION

The present invention provides (−)-3,3,3-trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester.

All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of the compound of the present invention that is compounds which are converted into the compound of the invention in vivo.

Pharmaceutical Preparations

The compound of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compound of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including the compound of the invention, or pharmaceutically acceptable derivatives thereof, in admixture with pharmaceutically acceptable adjuvants, diluents and/or carriers.

Pharmacological Properties

The compound of the invention is useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compound of the invention is also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compound is also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compound is also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compound is also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compound is also potentially useful as agents in treatment of (esophageal) achalasia.

In another aspect the present invention provides the compound of the invention for use as a medicament.

In a further aspect the present invention provides the use of a compound of the invention in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of the invention in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of the compound of the invention in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of the compound of the invention in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of the compound of the invention in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disoerders (e.g. arthritis deformans, inflammation, inflammatory sequelae of viral encephalitis, osteoarthritis) and orthopedic disorders.

In a still further aspect the present invention provides a method comprising administering a pharmacologically effective amount of the compound of the invention to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of the compound of the invention to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse. In a still further aspect the present invention provides a method comprising administering a pharmacologically effective amount of the compound of the invention to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma. In a still further aspect the present invention provides a method comprising administering a pharmacologically effective amount of the compound of the invention is to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of the compound of the invention to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compound of the present invention is particulary suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compound of the invention is useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compound is also potentially useful for the treatment of immune, cardiovascular, reproductive and endocrine disorders, septic shock and diseases related to the respiratory and gastrointestinal systems (e.g. diarrhea). The compound is also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compound may also eliminate the increase in weight that normally accompanies the cessation of smoking.

In a further aspect the present invention provides the use of the compound of the invention in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of the compound of the invention to a patient in need thereof.

The compound of the present invention is particulary suitable for the treatment of obesity, e.g. by reduction of appetite and body weight, maintenance of weight reduction and prevention of rebound.

The compound of the present invention may also be used to prevent or reverse medication-induced weight gain, e.g. weight gain caused by antipsychotic (neuroleptic) treatment(s). The compound of the present invention may also be used to prevent or reverse weight gain associated with smoking cessation.

The compound of the present invention is suitable for use in treating the above indications in juvenile or adolescent patient populations.

The compound of the present invention may also be suitable for use in the regulation of bone mass and bone loss and therefore useful in the treatment of osteoporosis and other bone diseases.

The compound of the present invention may also be used in the treatment of hepatic diseases, for example hepatic fibrosis, alcoholic liver cirrhosis, chronic viral hepatitis, non-alcoholic steatohepatitis or liver cancer.

Combination Therapy

The compound of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compound of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, the compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compound of the invention may also be combined with therapeutic agents used to treat complications related to micro-angiopathies.

The compound of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of the invention may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the compound of the invention may be used in conjunction with a sulfonylurea. The present invention also includes the compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes the compound of the present invention in combination with an inhibitor of the ileal bile acid transport system (IBAT inhibitor). The present invention also includes the compound of the present invention in combination with a bile acid binding resin.

The present invention also includes the compound of the present invention in combination with a bile acid sequestering agent, for example colestipol or cholestyramine or cholestagel.

According to an additional further aspect of the present invention there is provided a combination treatment comprising the administration of an effective amount of the compound of the invention optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products;

a phytosterol compound;

probucol;

an anti-coagulant;

an omega-3 fatty acid;

another anti-obesity compound for example sibutramine, phentermine, orlistat, bupropion, ephedrine, thyroxine;

an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;

a melanin concentrating hormone (MCH) modulator;

an NPY receptor modulator;

an orexin receptor modulator;

a phosphoinositide-dependent protein kinase (PDI) modulator; or

modulators of nuclear receptors for example LXR, FXR, RXR, GR, ERRα, β, PPARα, β, γ and RORalpha;

a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAUI), a tricyclic antidepressive agent (TCA), a noradrenergic and specific serotonergic antidepressant (NaSSA);

an antipsychotic agent for example olanzapine and clozapine;

a serotonin receptor modulator;

a leptin/leptin receptor modulator;

a ghrelin/ghrelin receptor modulator;

a DPP-IV inhibitor;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of the compound of the invention optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of the compound of the invention in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of the compound of the invention in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises the compound of the invention and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising the compound of the invention and a compound from one of the other classes of compounds described in this combination section or a pharmaceutically acceptable salt, solvate, solvate of such a salt or a prodrug thereof.

According to a further aspect of the present invention there is provided a kit comprising:

a) the compound of the invention in a first unit dosage form;

b) a compound from one of the other classes of compounds described in this combination section or a pharmaceutically acceptable salt, solvate, solvate of such a salt or a prodrug thereof; in a second unit dosage form; and

c) container means for containing said first and second dosage forms.

According to a further aspect of the present invention there is provided a kit comprising:

a) the compound of the invention together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of the compound of the invention and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of the compound of the invention and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of the compound of the invention optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

As the compound of the invention is useful in causing smoking cessation, preventing weight gain resulting from smoking cessation, treating nicotine withdrawal and preventing nicotine dependence they may also be combined with other compounds known to have one or more of these effects for example nicotine, a nicotine agonist or a partial agonist, a monoamine oxidase inhibitor or antidepressants such as bupropion, doxepine, nortriptyline or an anxiolytic such as buspirone or clonidine.

Pharmacological Activity

The compound of the present invention is active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x) ÙD)) and the IC50 value determined as the concentration required to give half maximal inhibition of GTPγS binding under the conditions used.

The compound of the present invention are active at the CB1 receptor (IC50<1 micromolar). For example, Example 1 has an IC50 of 2.3 nM.

The compound of the invention is believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of the compound of the present invention in models of gastrointestinal and/or cardiovascular function indicates that they offer significant advantages compared to representative reference CB1 antagonist/inverse agonist agents.

The compound of the present invention may provide additional benefits in terms of physical properties, potency, selectivity profile, bioavailability, half-life in plasma, blood brain permeability, plasma protein binding (for example higher free fraction of drug) or solubility compared to representative reference CB1 antagonists/inverse agonist agents. The utility of the compound of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### Examples

Abbreviations
AcOH acetic acid
AIBN 2,2′-azobisisobutyronitrile
BOP benzotriazol-1-yloxytris(dimethylamino)phosphonium hexafluorophosphate
DCM dichloromethane
DMF dimethylformamide
DEA diethylamine
DEAD diethyl azodicarboxylate
DIEA N,N-diisopropylethylamine
DMAP 4-dimethylaminopyridine
DMF N,N-dimethylformamide
EtOAc ethyl acetate
LiHMDS lithium hexamethyldisilazide
MeOH methanol
rt or RT room temperature
TEA triethylamine
THF tetrahydrofuran
TLC thin layer chromatography
t triplet
s singlet
d doublet
q quartet
qvint quintet
m multiplet
br broad
bs broad singlet
dm doublet of multiplet
bt broad triplet
dd doublet of doublet

General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC (High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5). For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

### Example 1

### (−)3,3,3-Trifluoro-propane-1-sulfonic acid 4-[2-(2,4-dichloro-phenyl)-5-(2-hydroxy-cyclohexylcarbamoyl)-4-hydroxymethyl-2H-pyrazol-3-yl]-phenyl ester

### Step A 1-(2,4-Dichlorophenyl)-5-(4-methoxyphenyl)-1-methyl-1H-pyrazole-3-carboxylic acid ethyl ester

To a magnetically stirred solution of lithium bis(trimethylsilyl)amide (200 ml, 1 M solution in hexane, 0.2 mol) in ether (600 ml) was added a solution of 4-methoxypropiophenone (32.84 g, 0.20 mol) in ether (200 ml) at −78° C. After stirring at this temperature for further 45 mins diethyl oxalate (34.5 g, 0.235 mol) was added and the reaction mixture stirred at room temperature overnight. The precipitated material was collected by filtration, washed with ether and dried to afford 30.81 g (57%) of the lithium salt as a pale yellow solid. To a magnetically stirred solution of this lithium salt (30.81, 0.11 mol) in 450 ml ethanol was added 2,4-dichlorophenylhydrazine hydrochloride (46.9 g, 0.22 mol) and the resulting mixture stirred at room temperature overnight. The precipitated material was filtered off, dried, dissolved in acetic acid and boiled under reflux overnight. Ice-water was added and the product extracted with EtOAc (×3). The combined organic extracts were washed with water, dried (Na₂SO₄), filtered and concentrated. Flash chromatography (Heptane: EtOAc gradient) afforded 19.4 g (24%) of the title compound.

Step B 1-(2,4-Dichloro-phenyl)-5-(4-hydroxyphenyl)-4-methyl-1H-pyrazole-3-carboxylic acid. To a solution of 1-(2,4-dichloro-phenyl)-5-(4-methoxyphenyl)-4-methyl-1H-pyrazole-3-carboxylic acid, (3.34 g, 8.85 mmol) in acetic acid was added 48% HBr (aq) (8.5 ml) dropwise and the reaction mixture refluxed overnight. After cooling to room temperature the reaction mixture was poured onto ice-water and extracted with EtOAc (×3). The combined organic extracts were washed with water, NaHCO₃ (aq) and brine. Drying (Na₂SO₄), filtration and concentration left 3.00 g (93%) of the title compound as a colorless solid.

### Step C 1-(2,4-Dichlorophenyl)-5-(4-hydroxyphenyl)-4-methyl-1H-pyrazole-3-carboxylic acid methyl ester

A solution of 1-(2,4-dichlorophenyl)-5-(4-hydroxyphenyl)-4-methyl-1H-pyrazole-3-carboxylic acid (0.36 g, 1.00 mmol) in 1.25 M HCl in methanol (10 ml) was refluxed for 1.5 hours. After cooling to room temperature, water was added and the product extracted with EtOAc (×3). The combined organic extracts were washed with water, dried (Na₂SO₄), filtered and concentrated. Flash chromatography (heptane:EtOAc 70:30-50:50) afforded 0.32 g (85%) of the title compound as a colorless solid.

### Step D 5-[4-(tert-Butyldimethyl-silanyloxy)phenyl]-1-(2,4-dichloro-phenyl)-4-methyl-1H-pyrazole-3-carboxylic acid methyl ester

To a solution of 1-(2,4-dichlorophenyl)-5-(4-hydroxyphenyl)-4-methyl-1H-pyrazole-3-carboxylic acid methyl ester (2.69 g, 7.13 mmol) in DMF (50 ml) was added imidazole (0.98 g, 14.3 mmol) followed by t-butyldichlorodimethylsilane (2.15 g, 14.3 mmol). The reaction mixture was stirred at room temperature overnight, diluted with water and extracted with ether (×2). The combined organic extracts were washed with water, dried (Na₂SO₄), filtered and concentrated. Purification by flash chromatography (heptane:EtOAc gradient) afforded 2.50 g (71%) of the title compound as a colorless solid.

### Step E 4-Bromomethyl-5-[4-(tert-butyl-dimethyl-silanyloxy)-phenyl]-1-(2,4-dichloro-phenyl)-1H-pyrazole-3-carboxylic acid methyl ester

To a solution of 5-[4-(tert-butyl-dimethylsilanyloxy)-phenyl]-1-(2,4-Dichloro-phenyl)-4-methyl-1H-pyrazole-3-carboxylic acid methyl ester, (3.70 g, 7.53 mmol) in 1,2-dichloroethane (120 ml) was added N-bromosuccinimide (1.51 g, 8.48 mmol) and a catalytic amount of AIBN. The reaction mixture was heated at reflux for one hour, cooled to room temperature, water was added and the product extracted with DCM (×2). The combined organic extracts were dried (Na₂SO₄), filtered and concentrated. Chromatography (heptane:EtOAc gradient) affording 4.15 g (96%) of the title compound as a colorless solid.

### Step F 1-(2,4-Dichlorophenyl)-4-hydroxymethyl-5-(4-hydroxyphenyl)-1H-pyrazole-3-carboxylic acid methyl ester

To a suspension of 4-bromomethyl-5-[4-(tert-butyldimethylsilanyloxy)phenyl]-1-(2,4-dichlorophenyl)-1H-pyrazole-3-carboxylic acid methyl ester (4.15 g, 7.27 mmol) in acetone:water (40:40 ml) was added silver nitrate (4.32 g, 25.4 mmol) and the reaction mixture stirred at 60° C. overnight, cooled to rt, filtered and after addition of water extracted with DCM (×2). The combined organic extracts were washed with brine, dried (Na₂SO₄), filtered and concentrated to afford 2.47 g (87%) of the title compound as a colorless solid after flash chromatography (heptane:EtOAc 50:50).

### Step G 1-(2,4-Dichlorophenyl)-4-hydroxymethyl-5-[4-(3,3,3-trifluoropropane-1-sulfonyloxy)phenyl]-1H-pyrazole-3-carboxylic acid methyl ester

To a solution of 1-(2,4-dichlorophenyl)-4-hydroxymethyl-5-(4-hydroxyphenyl)-1H-pyrazole-3-carboxylic acid methyl ester (980 mg, 2.50 mmol) in dichloromethane (10 ml) was added triethylamine (0.42 ml, 3.00 mmol) at 0° C. followed by 3,3,3-trifluoropropanesulfonyl chloride (589 mg, 3.00 mmol) in dichloromethane (10 ml). The reaction mixture was stirred at 0° C. for 2 hours, poured into water, extracted with dichloromethane (×3), the combined organic extracts dried (Na₂SO₄), filtered and concentrated. Flash chromatography (heptane:EtOAc 70:30-50:50) afforded 0.77 g (56%) of the product as a colorless solid.

### Step H 1-(2,4-Dichlorophenyl)-4-hydroxymethyl-5-[4-(3,3,3-trifluoro-propane-1-sulfonyloxy)phenyl]-1H-pyrazole-3-carboxylic acid

To a solution of 1-(2,4-dichlorophenyl)-4-hydroxymethyl-5-[4-(3,3,3-trifluoropropane-1-sulfonyloxy)phenyl]-1H-pyrazole-3-carboxylic acid methyl ester (0.77 g, 1.39 mmol) in MeOH:THF (10 ml:10 ml) at 0° C. was added a solution of lithium hydroxide (0.23 g, 5.60 mmol) in water (10 ml). The reaction mixture was stirred at this temperature for 1.5 hrs, acidified with 1M HCl to pH 3 and the product extracted with EtOAc (×3). The combined organic extracts were washed with water, dried (Na₂SO₄), filtered and concentrated. Flash chromatography (heptane:EtOAc 50:50) afforded 0.70 g (93%) of the title compound as a colorless solid.

### Step I 3,3,3-Trifluoropropane-1-sulfonic acid 4-[2-(2,4-dichlorophenyl)-5-(2-hydroxy-cyclohexylcarbamoyl)-4-hydroxymethyl-2H-pyrazol-3-yl]phenyl ester

To a suspension of 1-(2,4-dichlorophenyl)-4-hydroxymethyl-5-[4-(3,3,3-trifluoropropane-1-sulfonyloxy)phenyl]-1H-pyrazole-3-carboxylic acid (0.70 g, 1.30 mmol) in dichloromethane (90 ml) was added cis-2-aminocyclohexanol hydrochloride (200 mg, 1.32 mmol) followed by triethylamine (0.37 ml, 2.63 mmol) and BOP (708 mg, 1.60 mmol). The reaction mixture was stirred at room temperature overnight, poured into ice-water and extracted with EtOAc (×3). The combined organic extracts were dried (Na₂SO₄), filtered and concentrated. Flash chromatography (heptane:EtOAc 50:50-EtOAc) and CH₂Cl₂:MeOH 99:1-95:5) afforded 200 mg (24%) of the title compound as a colorless solid. HPLC: 89%. MS: 658 (M+Na).

Step J Chiral chromatography (Chiralpak AD, heptane/ethanol 70/30) on (+/−)3,3,3-Trifluoro-propane-1-sulfonic acid 4-[2-(2,4-dichloro-phenyl)-5-(2-hydroxy-cyclohexylcarbamoyl)-4-hydroxymethyl-2H-pyrazol-3-A-phenyl ester from Ex. 1, Step I (1.10 g, 1.73 mmol) resulted in (−)3,3,3-Trifluoro-propane-1-sulfonic acid 4-[2-(2,4-dichloro-phenyl)-5-(2-hydroxy-cyclohexylcarbamoyl)-4-hydroxymethyl-2H-pyrazol-3-yl]-phenyl ester (553 mg, 50.3%, 98.8% ee) obtained as a solid.

[α]_{D}²: −=12.3 (c 1.0, chloroform)

¹H-NMR (400 MHz, CDCl₃) 7.39-7.32 (2H, m), 7.28-7.12 (6H, m), 5.16 (1H, t), 4.54 (2H, d), 4.11-3.97 (2H, m), 3.45-3.38 (2H, m), 2.78-2.65 (2H, m), 1.88-1.83 (1H, m), 1.78-1.31 (8H, m).

HRMS Calcd for [C₂₆H₂₆Cl₂F₃N₃O₆S+H]⁺: 636.095. Found: 636.094.

1. The compound (−)-3,3,3-trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester.

2. (canceled)

3. A pharmaceutical formulation comprising a compound as claimed in claim 1 and a pharmaceutically acceptable adjuvant, diluent or carrier.

4. (canceled)

5. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound as claimed in claim 1 to a patient in need thereof.

6. (canceled)

7. A process for the preparation of a compound as claimed in claim 1 comprising separating racemic 3,3,3-trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester by chiral chromatography.
